(19) United States
(12) Patent Application Publication
(10) Pub. No.: US 2022/0233231 A1
(43) Pub. Date: Jul. 28, 2022

(54) FALLOPIAN TUBE SEALING DEVICE

(71) Applicant: Covidien LP, Mansfield, MA (US)

(72) Inventors: Nikolai D. Begg, Wellesley, MA (US); Archana Penumudi, San Diego, CA (US); Natalija Beslic, Brookline, MA (US)

(21) Appl. No.: 17/560,341

(22) Filed: Dec. 23, 2021

Related U.S. Application Data
(60) Provisional application No. 63/140,375, filed on Jan. 22, 2021.

(51) Int. Cl. A61B 18/14; A61B 18/12

(52) U.S. Cl. CPC A61B 18/14; A61B 2018/0063; A61B 18/1206

(57) ABSTRACT

A device for sealing tissue within an anatomical structure includes a handle having an elongated shaft extending therefrom. A reciprocating element is disposed within the elongated shaft and is configured for reciprocation therein. The reciprocating element includes a distal end extending from a distal end of the elongated shaft. A rope-like filament is secured to the distal end of the reciprocating element and remains exposed relative to the distal end of the elongated shaft during reciprocation thereof. A motor is disposed within the handle and is configured to induce reciprocation of the reciprocating element upon activation thereof, the motor is adapted to connect to a generator configured to selectively supply power to the motor upon activation thereof.

### CROSS-REFERENCE TO RELATED APPLICATION

This application claims the benefit of and priority to U.S. Provisional Patent Application No. 63/140,375 filed Jan. 22, 2021, the entire disclosure of which is incorporated by reference herein.

### BACKGROUND

### 1. Technical Field

The present disclosure relates to surgical devices and, more specifically, devices and methods for sealing fallopian tubes.

### 2. Discussion of Related Art

Due to a history of pelvic disease, adhesions, tubal disease or hydrosalpinx, many women experience difficulty conceiving. As a result, in vitro fertilization (IVF) has become a popular alternative to traditional conception. One of the inherent risks associated with IVF is the occurrence of an ectopic pregnancy wherein the fertilized egg(s) implants within the fallopian tube instead of the uterus. In the case of hydrosalpinx an abnormal buildup of fluid may affect IVF success.

Surgical procedures such as salpingectomies or laparoscopic tubal ligations are typically performed to lower the risk of ectopic pregnancies and to minimize the effects of unilateral or bilateral hydrosalpinx. As with all procedures, these have their own risks and associated complications. Sealing the fallopian tubes may be an alternative solution with minimal risk.

### SUMMARY

This disclosure relates generally to surgical devices that can be used through a hysteroscopic approach to seal fallopian tubes without leaving foreign materials within the body cavity. In addition, the surgical devices disclosed herein may be used in a medical office without the need for general anesthesia.

The present disclosure relates to a device for sealing tissue within an anatomical structure (e.g., fallopian tube) and includes a handle having an elongated shaft extending therefrom. A reciprocating element is disposed within the elongated shaft and is configured for reciprocation therein, the reciprocating element including a distal end extending from a distal end of the elongated shaft. A rope-like filament is secured to the distal end of the reciprocating element, the rope-like filament remaining exposed relative to the distal end of the elongated shaft during reciprocation thereof. A motor is disposed within the handle and is configured to induce reciprocation of the reciprocating element upon activation thereof. The motor is adapted to connect to a generator configured to selectively supply power to the motor upon activation thereof.

In aspects according to the present disclosure, a switch is operably coupled to the motor and is operably associated with the handle. In other aspects according to the present disclosure, the rope-like filament is wound around the distal end of the reciprocating element. In yet other aspects according to the present disclosure, the rope-like filament is void of any sharp edges.

In aspects according to the present disclosure, the motor cooperates with a cam to induce reciprocating motion of the reciprocating element. In other aspects according to the present disclosure, the cam is disposed within the elongated shaft. In still other aspects according to the present disclosure, the cam is disposed within the handle.

The present disclosure relates to a method for sealing tissue within an anatomical structure and includes reciprocating a rope-like filament within a fallopian tube to induce an inflammatory response in the tissue. The rope-like filament is disposed on a distal end of a reciprocating element disposed within an elongated shaft extending from a handle. The reciprocating element is operably connected to a motor disposed within the handle and is configured to induce reciprocation of the reciprocating element upon activation thereof. The motor is adapted to connect to a generator configured to selectively supply power to the motor upon activation thereof.

In aspects according to the present disclosure, the method further includes a switch operably coupled to the motor and disposed in the handle. In other aspects according to the present disclosure, the method includes winding the rope-like filament around the distal end of the reciprocating element.

In aspects according to the present disclosure, the rope-like filament is void of any sharp edges. In other aspects according to the present disclosure, the motor cooperates with a cam to induce reciprocating motion of the reciprocating element. In still other aspects according to the present disclosure, the cam is disposed within the elongated shaft. In yet other aspects according to the present disclosure, the cam is disposed within the handle.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a side view of an exemplary electrosurgical system provided in accordance with the present disclosure including a surgical device and a generator;

FIGS. 2A-2C are enlarged, schematic views a rope or rope-like filament being secured to a distal end the surgical device; and

FIGS. 3A-3B are schematic views of the surgical device shown reciprocating within a fallopian tube to induce an inflammatory response to seal the tube.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closer to the clinician and the term “distal” refers to the portion of the device or component thereof that is farther from the clinician.

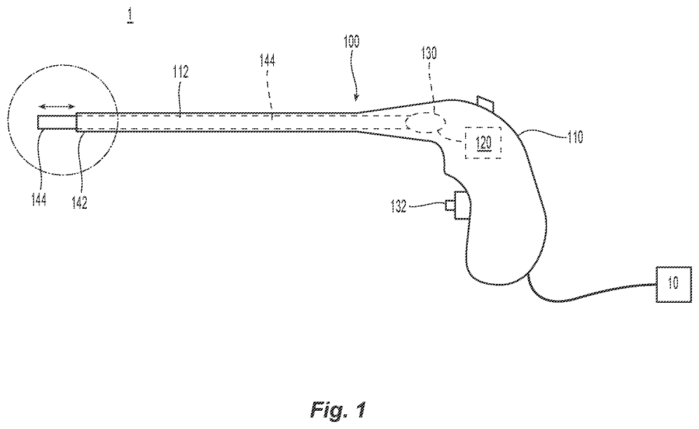

Referring now to FIG. 1, an electrosurgical system 1 is disclosed in accordance with the present disclosure and includes a generator 10 and a surgical device 100 including an end effector 140 couple to a distal end thereof. The generator 10 is configured to provide energy to the surgical device 100. As detailed herein, the energy is provide to a motor to induce a reciprocating motion to the end effector of the surgical device 100.

The surgical device 100 includes a handle 110, an elongated body or shaft 112 extending from the handle 110, and an end effector 140 supported by the distal portion of the elongated shaft 112. The elongated shaft 112 may be flexible, semi-rigid, or rigid. In embodiments the elongated shaft 112 is curved along a longitudinal axis thereof to aid in insertion into a fallopian tube “FT” as detailed below.

End effector 140 includes a distal end 142 having a reciprocating element 144 disposed therein. Reciprocating element 144 is configured to extend from the distal end 142 and reciprocate within shaft 112 upon activation of a switch 132. Switch 132 communicates with a motor 120 which, in turn, electrically couples to a cam 130 to induce reciprocation of reciprocating element 144 upon movement thereof. Other mechanisms are envisioned to induce reciprocation of reciprocating element 144, e.g., pneumatic mechanisms, linkages, oscillators, solenoids, etc. Cam 130 may be disposed within handle 110 or be housed more distally within the instrument 100 or elongated shaft 112.

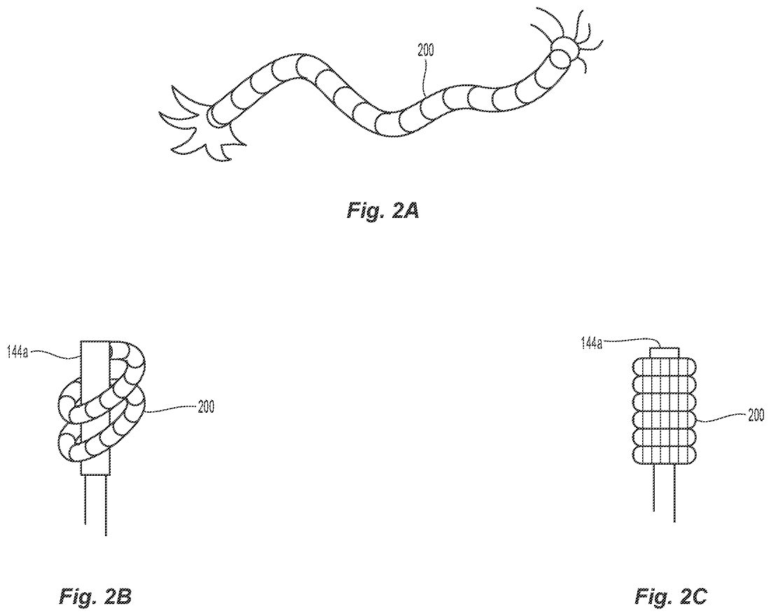

Turning now to FIGS. 2A-2C, prior to use, a distal end 144a of the reciprocating element 144 is extended to its distalmost position. A rope or rope-like filament 200 is wound around or otherwise secured to the distal end 144a of the reciprocating element 144. Typically, only the portion of the distal end 144a of the reciprocating element 144 is wound with the rope 200 so as not to inadvertently separate the rope 200 from the distal end 144a.

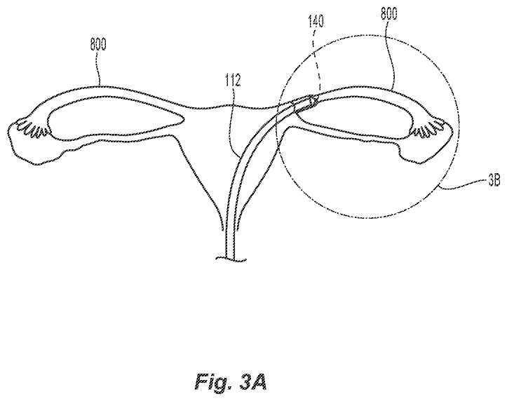

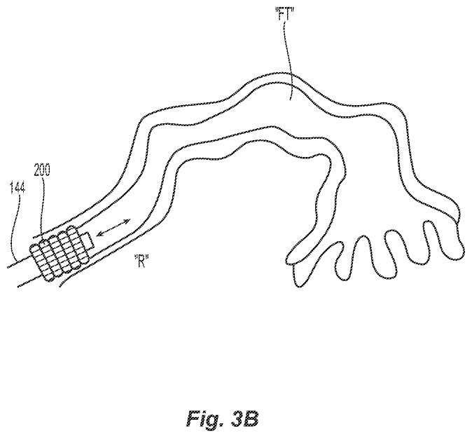

Once the rope 200 is secured about the distal end 144a of the reciprocating element 144, the elongated shaft 112 may be inserted into the fallopian tube “FT” of a patient as shown in FIGS. 3A and 3B. The flexibility of the elongated shaft 112 facilitates positioning of the distal end 144a and rope 200 proximate a desired area of treatment. Once inserted, the surgeon activates switch 132 which, in turn, activates motor 120 to rotate the cam 130 to reciprocate the reciprocating element 144 within shaft 112 causing the rope 200 to irritate, heat and burn the tissue proximate the distal end 144a.

Using heat generated by the friction or abrasion of the rope 200 against the tissue of the tube “FT”, the rope 200 essentially burns the tissue which, in turn, induces an inflammatory response and the formulation of granulated tissue. As a result, the tube “FT” seals under the inflammatory response effectively blocking the tube “FT” and preventing a fertilized egg associated ectopic pregnancy or hydrosalpinx fluid from entering therein. By using a strictly mechanical approach to tissue response, potential complications from electrosurgical treatment of tissue (e.g., bipolar or monopolar) are avoided. Moreover, the rope 200 is void of sharp edges or areas of potential electrical arcing further mitigating areas of potential surgical concern with perforation of the tube “FT”.

The present disclosure relates to a method for sealing tissue within a fallopian tube “FT” and includes reciprocating a rope-like filament 200 within a fallopian tube “FT” to induce an inflammatory response in the tissue. The rope-like filament 200 is disposed on a distal end 144a of a reciprocating element 144 disposed within an elongated shaft 112 extending from a handle 110. The reciprocating element 144 is operably connected to a motor 120 disposed within the handle 110 and is configured to induce reciprocation of the reciprocating element 144 upon activation thereof. The motor 120 is adapted to connect to a generator 10 configured to selectively supply power to the motor 120 upon activation thereof.

Sealing a fallopian tube “FT” with instrument 100 may be performed in a medical office without requiring general anesthesia and may be completed without leaving implanted or foreign materials within the patient. Further, the method of sealing the fallopian tube “FT” detailed above forms an immediate and temporary seal (or possibly a permanent seal) which may reduce the time of a subsequent surgical procedure, reduce the cost of a subsequent surgical procedure, and/or improve patient outcomes associated with sealing fallopian tubes.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A device for sealing tissue within a fallopian tube, comprising:
a handle having an elongated shaft extending therefrom;
a reciprocating element disposed within the elongated shaft and configured for reciprocation therein, the reciprocating element including a distal end extending from a distal end of the elongated shaft;
a rope-like filament secured to the distal end of the reciprocating element, the rope-like filament remaining exposed relative to the distal end of the elongated shaft during reciprocation thereof; and
a motor disposed within the handle and configured to induce reciprocation of the reciprocating element upon activation thereof, the motor adapted to connect to a generator configured to selectively supply power to the motor upon activation thereof.

2. The device for sealing tissue according to claim 1, further comprising a switch operably coupled to the motor and operably associated with the handle.

3. The device for sealing tissue according to claim 1, wherein the rope-like filament is wound around the distal end of the reciprocating element.

4. The device for sealing tissue according to claim 1, wherein the rope-like filament is void of any sharp edges.

5. The device for sealing tissue according to claim 1, wherein the motor cooperates with a cam to induce reciprocating motion of the reciprocating element.

6. The device for sealing tissue according to claim 5, wherein the cam is disposed within the elongated shaft.

7. The device for sealing tissue according to claim 5, wherein the cam is disposed within the handle.

8. A method for sealing tissue within a fallopian tube, comprising:
reciprocating a rope-like filament within a fallopian tube to induce an inflammatory response in the tissue, the rope-like filament disposed on a distal end of a reciprocating element disposed within an elongated shaft extending from a handle, the reciprocating element operably connected to a motor disposed within the handle and configured to induce reciprocation of the reciprocating element upon activation thereof, the motor adapted to connect to a generator configured to selectively supply power to the motor upon activation thereof.

9. The method for sealing tissue according to claim 8, further comprising a switch operably coupled to the motor and disposed in the handle.

10. The method for sealing tissue according to claim 8, further comprising winding the rope-like filament around the distal end of the reciprocating element.

11. The method for sealing tissue according to claim 8, wherein the rope-like filament is void of any sharp edges.

12. The method for sealing tissue according to claim 8, wherein the motor cooperates with a cam to induce reciprocating motion of the reciprocating element.

13. The method for sealing tissue according to claim 12, wherein the cam is disposed within the elongated shaft.

14. The method for sealing tissue according to claim 12, wherein the cam is disposed within the handle.
